(19) United States
(12) Patent Application Publication
(10) Pub. No.: US 2025/0208054 A1
(43) Pub. Date: Jun. 26, 2025

(54) ARTIFICIAL LYOPHILIZED PRODUCT SAMPLES FOR AUTOMATED VISUAL INSPECTION SYSTEMS

(71) Applicant: AMGEN INC., Thousand Oaks, CA (US)

(72) Inventor: Thomas Clark Pearson, Newbury Park, CA (US)

(21) Appl. No.: 18/850,407

(22) PCT Filed: Mar. 24, 2023
(86) PCT No.: PCT/US23/16179

§ 371 Date: Sep. 24, 2024

Related U.S. Application Data
(60) Provisional application No. 63/323,688, filed on Mar. 25, 2022.

(51) Int. Cl. G01N 21/88; B01L 3/00; G01N 21/90; G01N 33/15

(52) U.S. Cl. CPC G01N 21/8803; B01L 3/50825; G01N 21/90; G01N 33/15; B01L 2200/16

(57) ABSTRACT

Artificial lyo-vials are provided that include a portion of a vial. The portion of the vial includes a vial wall with a top end opening and a bottom end opening. The bottom end opening includes an inside diameter that is greater than an inside diameter of the top end opening. At least a portion of the vial wall is translucent. The artificial lyo-vials include an artificial lyo-cake. The artificial lyo-cake is secured within the portion of the vial. The artificial lyo-cake includes a base, an annular surface, a top surface, and a longitudinal dimension extending from the base to at least a portion of the top surface. An outside diameter of the artificial lyo-cake is greater than the inside diameter of the top end opening.

### FIELD OF DISCLOSURE

The present application relates generally to the inspection of lyophilized pharmaceutical products, and more specifically to the creation and use of artificial lyophilized product samples for purposes of training, refining, and/or qualifying automated visual inspection systems.

### BACKGROUND

In certain contexts, such as quality control procedures for manufactured drug products, it is necessary to examine samples (e.g., lyophilized product samples) for the presence of various defects (e.g., low fill, high fill, collapsed cake, meltback, foreign particles, fibers, etc.). The acceptability of a given sample, under the applicable quality standards, may depend on metrics such as a condition of the lyophilized product, the presence of undesired particles contained within the sample, etc. If a sample has unacceptable metrics, it may be rejected and discarded.

To handle the quantities typically associated with commercial production of pharmaceuticals, the product inspection tasks have increasingly become automated. However, automated visual inspection (AVI) systems have struggled to overcome various barriers to achieving good product fidelity void of system complexities. For example, inspecting lyophilized pharmaceutical products, which are often distributed in glass vials, for defects is one of the most difficult challenges in an AVI process. One reason for the difficulty with known AVI systems is that lyophilized drug products degrade over time. Testing and tuning AVI systems for inspection of lyophilized products has an added challenge in that a lyophilized product cake (“lyo-cake”) structure often has a short lifetime due to the agitation the lyo-cake is exposed to in the AVI system. Thus, lyo-samples can typically only be run through an AVI system a few times before the lyo-cake sample loses an associated structure. Consequentially, vials containing a lyophilized product (i.e., “lyo-vials”) cannot reliably and repeatedly be used to test or tune an AVI system. Instead, new samples need to be made, adding cost and/or causing delays. Moreover, manufacturing lyo-cakes with defects can be expensive, and can slow down development, characterization, and/or testing of AVI equipment.

### SUMMARY

Embodiments described herein relate to artificial lyophilized product cakes and modified vials containing artificial lyophilized product cakes.

As described herein, an artificial lyo-vial is representative of at least a portion of a lyophilized product sample. The artificial lyo-vial includes a portion of a vial. The portion of the vial includes a vial wall with a top end opening and a bottom end opening. The bottom end opening of the vial includes an inside diameter that is greater than an inside diameter of the top end opening of the vial. At least a portion of the vial wall is translucent. An artificial lyo-cake is secured within the portion of the vial. The artificial lyo-cake includes a base, an annular surface, a top surface, a longitudinal dimension extending from the base to at least a portion of the top surface. An outside diameter of the artificial lyo-cake is greater than the inside diameter of the top end opening of the vial.

A method of manufacturing an artificial lyo-vial includes providing a vial including a top end opening having an top inside diameter. The method also includes creating a bottom end opening by removing at least a portion of a bottom end of the vial. The bottom end opening includes a bottom inside diameter that is greater than the top inside diameter. The method further includes providing an artificial lyo-cake having a base, a top surface, an annular surface extending between the base and the top surface. An outside diameter of the artificial lyo-cake is greater than the top inside diameter of the vial. The method yet further includes inserting at least a portion of the artificial lyo-cake through the bottom end opening of the vial.

A method of manufacturing an artificial lyo-cake includes generating three-dimensional data that defines a base of a lyo-cake, an annular surface of the lyo-cake, and a top surface of the lyo-cake. The method also includes receiving the three-dimensional data at a controller of a three-dimensional printer. The method further includes controlling material discharge from a material discharge device of the three-dimension printer, to dispense a material, based on at least z-values of the three-dimensional data. The method yet further includes controlling a position of the material discharge device relative to a platform based on at least x-values and y-values of the three dimensional data.

Novel artificial lyophilized product cakes and modified vials containing artificial lyophilized product cakes are provided. Novel methods for manufacturing artificial lyophilized product cakes and modified vials containing artificial lyophilized product cakes are also provided.

### BRIEF DESCRIPTION OF THE DRAWINGS

The skilled artisan will understand that the figures described herein are included for purposes of illustration and do not limit the present disclosure. The drawings are not necessarily to scale, and emphasis is instead placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 depicts an example automated visual inspection system for inspecting containers of lyophilized products.

FIGS. 2A through 2C depict an example artificial lyophilized cake (“artificial lyo-cake”) generation system and method.

FIGS. 3A through 3E depict an example method of generating an artificial vial (“artificial lyo-vial”) containing an artificial lyo-cake of FIGS. 2A-C.

FIG. 4 depicts an example artificial lyo-cake generated using the system and method of FIGS. 2A-C.

FIG. 5 depicts another example artificial lyo-cake generated using the system and method of FIGS. 2A-C.

FIGS. 6A through 6L depict grayscale images of example vials having defects associated with lyophilized products.

### DETAILED DESCRIPTION

The various concepts introduced above and discussed in greater detail below may be implemented in any of numerous ways, and the described concepts are not limited to any particular manner of implementation. Examples of implementations are provided for illustrative purposes.

As described in detail herein, lyophilized product defects such as high fill, low fill, and collapsed lyophilized product cake may be recreated using 3D printing to represent the lyophilized product cake (“artificial lyo-cake”). A texture and/or color of a lyophilized product may be recreated by, for example, coating a 3D printed artificial lyo-cake with sugars and salts diluted with isopropyl alcohol. Lyophilized product defects such as meltback, liquefaction, and/or foreign particles may be recreated by, for example, applying particles, textures and/or color variations to at least a portion of a top surface and/or at least a portion of an annular surface of an artificial lyo-cake.

Correspondingly, vials may be modified to accommodate the artificial lyo-cakes by cutting off at least a portion of a bottom of a vial and inserting the artificial lyo-cake through the bottom opening. When particle defects (e.g., glass particles, metallic particles, fibers, etc.) are to be represented in the artificial lyo-cake, actual particles may be placed on at least a portion of a top surface and/or at least a portion of an annular surface of the artificial lyo-cake before inserting the artificial lyo-cake into the bottom opening of the modified vial. The artificial lyo-cakes and artificial lyo-vials of the present disclosure simplify defect sample (e.g., low fill, high fill, collapsed cake, meltback, foreign particles, etc.) manufacturing. Additionally, the artificial lyo-cakes are robust and can survive, for example, shipping to facilities where AVI systems need to be trained and/or qualified. The artificial lyo-cakes and artificial lyo-vials may also be used to develop new lighting and inspection techniques for detecting glass particles. The artificial lyo-cakes and artificial lyo-vials may reduce costs, reduce delays, and/or improve inspection of lyophilized product by making samples representing specific defect types available on a fast, consistent, and reliable basis.

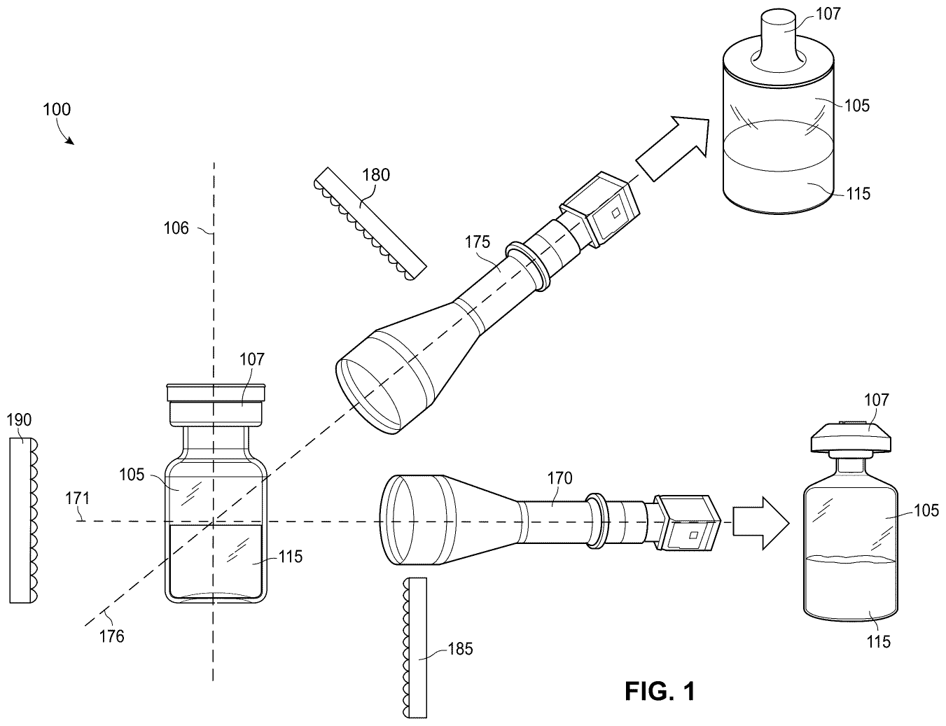

To provide context for use of the lyo-vials of the present disclosure, FIG. 1 depicts an automated visual inspection (AVI) system 100 for inspecting a vial 105 containing a lyophilized product 115. The example vial 105 has a central axis 106 and a seal 107. The example AVI system 100 also includes a profile view imager 170 having an optical axis 171, which may be oriented perpendicular to the central axis 106 of the container 105. As illustrated in FIG. 1, the profile view imager 170 may generate a profile view image of the vial 105, the seal 107, and the lyophilized product 115. As described in detail herein, profile view images of the vial 105 may be used in an AVI system 100 to detect a lyophilized product 115 fill level within a vial 105, a slope of a top surface of the lyophilized product 115 within the vial 105, and/or other characteristics of the lyophilized product 115.

The example AVI system 100 also includes a perspective view imager 175 located above the vial 105 and having an optical axis 176 aimed slightly downward toward the vial 105. As illustrated in FIG. 1, the perspective view imager 170 may generate a perspective view image of the vial 105, the seal 107, and the lyophilized product 115. As described in detail herein, perspective view images of the vial 105 may be used in an AVI system 100 to detect a shape of a top surface of the lyophilized product 115 within the vial 105, a texture of the top surface of the lyophilized product 115, a color of the top surface of the lyophilized product 115, and/or other characteristics of the top surface (and possibly surrounding areas) of the lyophilized product 115.

The AVI system 100 may include an angled light source 180, a direct light source 185, and/or a backlight source 190. As typical within AVI systems 100, activation of the angled light source 180, the direct light source 185, and/or the backlight source 190 may be coordinated with image acquisition from the profile view imager 170 and/or the perspective view imager 175 to increase contrast within resulting images of an artificial lyo-vial to, for example, detect defects in an associated artificial lyo-cake. In other embodiments, however, the AVI system 100 includes more, fewer, or differently positioned imagers (e.g., only profile view imager 170), and/or more, fewer, or differently positioned light sources (e.g., only angled light source 180).

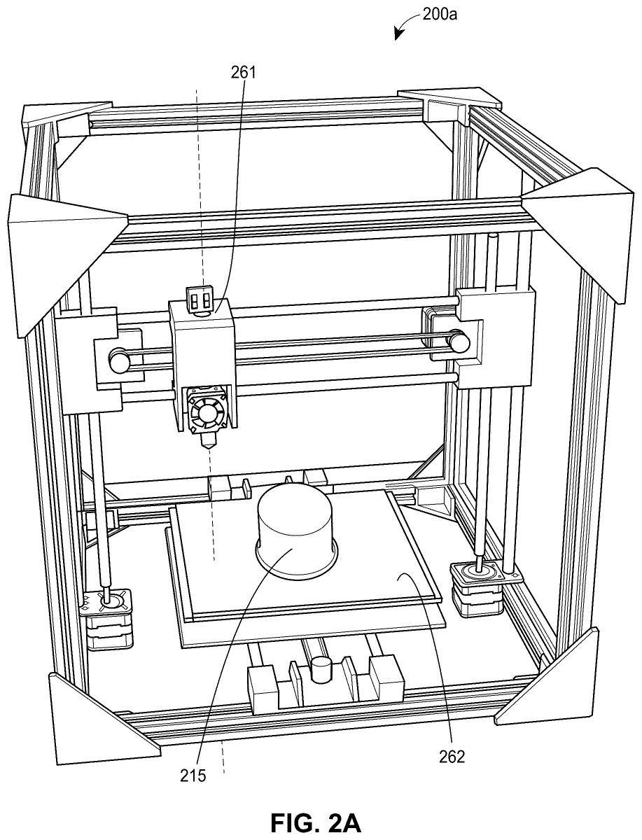

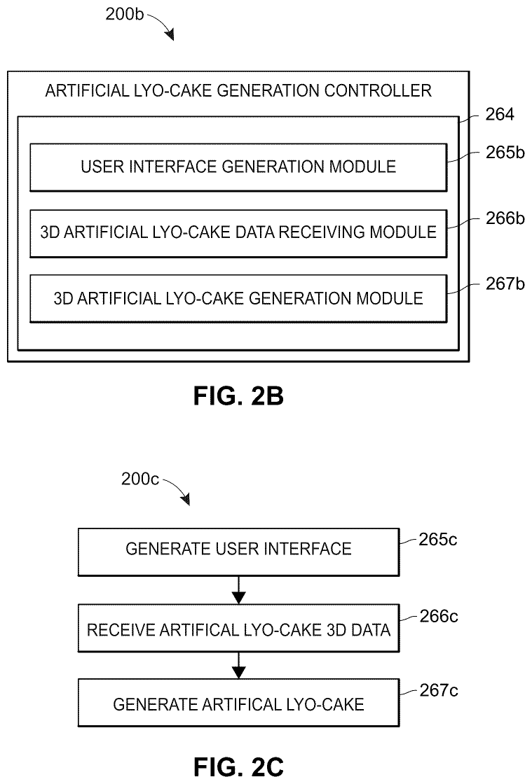

FIGS. 2A through 2C depict an artificial lyophilized cake (“artificial lyo-cake”) generation system (FIGS. 2A and 2B) and method (FIG. 2C). Referring first to FIG. 2A, the artificial lyo-cake generation system may include a three-dimensional (3D) printer 200a having a material dispenser 261 and a platform 262. As depicted in FIG. 2B, a controller 200b for the 3D printer 200a includes a user interface generation module 265b, a 3D artificial lyo-cake data receiving module 266b, and a 3D artificial lyo-cake generation module 267b. The user interface generation module 265b, the 3D artificial lyo-cake data receiving module 266b, and/or the 3D artificial lyo-cake generation module 267b may be, for example, computer-readable instructions stored on a non-transitory computer-readable medium 264. Alternatively, or additionally, at least a portion of the user interface generation module 265b, the 3D artificial lyo-cake data receiving module 266b, and/or the 3D artificial lyo-cake generation module 267b may be, for example, configured within an application specific integrated circuit (ASIC), a field programmable gate array (FPGA), firmware, or other dedicated electronic circuitry.

The controller 200b may execute at least portions of the user interface generation module 265b, the 3D artificial lyo-cake data receiving module 266b, and/or the 3D artificial lyo-cake generation module 267b to implement the method 200c. The controller 200b may execute the user interface generation module 265b to cause the controller 200b to generate a user interface (block 265c of FIG. 2C). The user interface may enable a user to configure, control and/or interact with the artificial lyo-cake generation system.

The controller 200b may execute the 3D artificial lyo-cake data receiving module 266b to cause the controller 200b to receive 3D artificial lyo-cake data (block 266c of FIG. 2C). The 3D artificial lyo-cake data may be, for example, formatted as a stereolithography (STL) file. The 3D artificial lyo-cake data may include Cartesian coordinates that define a base, an annular surface, and a top surface of an artificial lyo-cake 205. The 3D artificial lyo-cake data may be generated using a 3D laser scanner (i.e., scanning a lyophilized product using a 3D laser scanner). Alternatively, the 3D artificial lyo-cake data may be generated using computer aided design (CAD) software, and include special coordinates based on interior dimensions of a respective container along with coordinates that define a top surface of an artificial lyo-cake.

The controller 200b may execute the 3D artificial lyo-cake generation module 267b to cause the artificial lyo-cake generation system to generate an artificial lyo-cake (block 267c of FIG. 2C). For example, a 3D printer may generate an artificial lyo-cake based on the 3D artificial lyo-cake data. The controller 200b may control material discharge from the material discharge device 261 of the artificial lyo-cake generation system to dispense a material based on at least z-values of the three-dimensional data. The controller 263 controls a position of the material discharge device 261 relative to the platform 262 based on at least x-values and y-values of the three dimensional data.

At least a portion of a top surface and/or an annular surface of an artificial lyo-cake may be coated with, for example, sugars and salts diluted with isopropyl alcohol. The artificial lyo-cake may be manually coated. Alternatively, the controller 200b may further execute the 3D artificial lyo-cake generation module 267b to cause the artificial lyo-cake generation system to coat the artificial lyo-cake (block 267c), or (if resolution is sufficient) to cause the artificial lyo-cake to be generated in a manner that accurately reflects the desired surface textures/characteristics without coating.

A label may be, for example, manually added to an artificial lyo-cake and/or associated artificial lyo-cake packaging to identify a respective defect. Additionally, or alternatively, the controller 200b may further execute the artificial lyo-cake generation module 267b to add label data to the 3D artificial lyo-cake data (block 267c). The artificial lyo-cake label and/or label data may identify a respective artificial lyo-cake as acceptable, rejected, low-fill, high-fill, meltback, collapsed lyo-cake, lyo-cake with foreign particles, etc. The artificial lyo-cake label data may then be used to test and/or train (with supervised learning) an associated AVI system 100.

An artificial lyo-cake may be made from, for example, a semi-flexible material (e.g., thermoplastic polyurethane (TPU), etc.). Artificial lyo-cake defects, such as high fill, low fill, and collapsed cake, may be manufactured by 3D printing (e.g., using 3D printer 200a and controller 200b as discussed above). Other defects, such as meltback and artificial lyo-cake with foreign particles, can be made, for example, by coating a 3D printed artificial lyo-cake with sugars and/or salts that are diluted with isopropyl alcohol. When particle defects are to be made, respective particles may be placed on a top surface and/or an annular surface of the artificial lyo-cake.

As an alternative to 3D printing, an artificial lyo-cake may be manufactured using a computerized manufacturing process that implements computerized numerical control (CNC). As another alternative, an artificial lyo-cake may be manufactured using molding and/or co-molding techniques. An associated mold may be tooled based on the 3D artificial lyo-cake data. As another alternative, an artificial lyo-cake may be machined from a block of material. Relatedly, a controller may cause an associated machining apparatus to automatically machine the artificial lyo-cake based on the 3D artificial lyo-cake data.

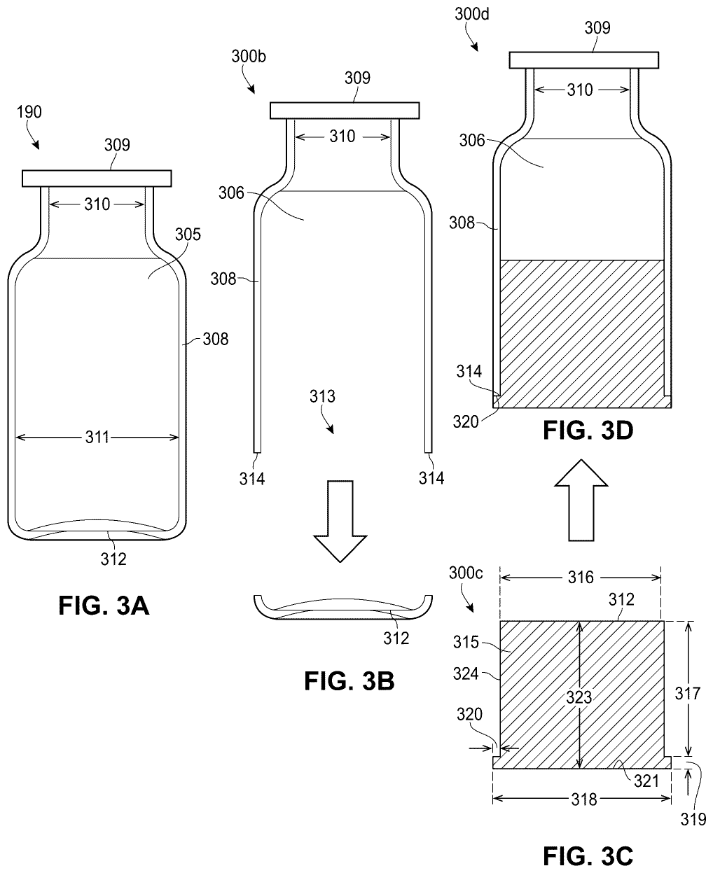

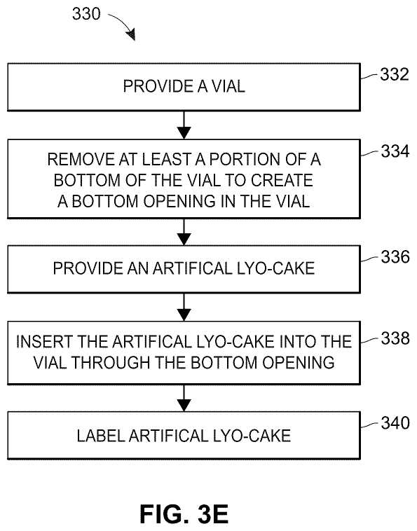

FIGS. 3A through 3D depict an example series of stages 300a-e for generating an artificial vial (“artificial lyo-vial”) containing an artificial lyo-cake, which correspond to method 334 of FIG. 3E. Stage 300a includes providing a vial 305 (block 338 of FIG. 3E). The vial 305 may include a top end opening 309 having an inside diameter 310 (and possibly covered by a cap), a translucent wall 308 having an inside diameter 311, and a bottom 312. The wall 308 and bottom 312 may be integrally formed of the same material (e.g., glass), for example.

Stage 300b includes removing the bottom 312 of the vial 305 to create a modified vial 306 having a bottom end opening 313 (block 334 of FIG. 3E). The modified vial 306 includes a bottom rim 314. In embodiments where the bottom 312 includes the entire base of the vial 305, the bottom rim 314 may be a portion of the wall 308 in which the vial sides are vertical (when the modified vial 305 is in an upright position). While the bottom end opening 313 is shown to have the same dimension as the vial inside diameter 311, the bottom end opening 313 may have a diameter that is different than the vial inside diameter 311 if the vial 305 is instead a non-uniformly shaped container. In any event, an inside diameter of the bottom end opening 313 is greater than the inside diameter 310 of the top end opening 309.

Stage 300c includes providing an artificial lyo-cake 315 (block 336 of FIG. 3E). The artificial lyo-cake 315 may be similar to the artificial lyo-cake 215 of FIG. 2, for example, and providing the artificial lyo-cake 315 may include using the 3D printer 200a to generate the artificial lyo-cake 315. The artificial lyo-cake 315 includes a base 321, an annular surface 324, a top surface 322, a longitudinal dimension 323 extending from the base 321 to at least a portion of the top surface 322, and an outside diameter 316 that is substantially greater than the inside diameter 310 of the top end opening 309. The artificial lyo-cake 315 may include an optional flange having an outside diameter 318, a height 319, and a width 320. When the artificial lyo-cake 315 includes the optional flange, the artificial lyo-cake 315 may include an additional longitudinal dimension 317. The flange may be integrally formed with the rest of the artificial lyo-cake 315 (e.g., also formed by the 3D printer 200a), or may be added at a later stage (e.g., a rubber or plastic cap that tightly fits on to, and or is glued to, the bottom end of the artificial lyo-cake 315).

Stage 300d includes inserting the artificial lyo-cake 315 into the modified vial 306 through the bottom end opening 313 (block 338 of FIG. 3E). When the artificial lyo-cake 315 includes a flange, the bottom rim 314 becomes proximate the flange width 320 when the artificial lyo-cake 315 is inserted into the bottom end opening 313. When the artificial lyo-cake 315 does not include an optional flange, the bottom rim 314 may be flush with the base 321.

In embodiments with and without the optional flange, the artificial lyo-cake 315 may fit snugly in the modified vial 306, such that friction helps retain the artificial lyo-cake 315 within the wall 308. That is, the means for securing the artificial lyo-cake 315 within the modified vial 306 may be the annular surface 324 and the artificial lyo-cake 315 themselves, dimensioned such that the latter fits snugly (by friction fit) into the modified vial 306, i.e., with the interior diameter 311 of the vial 305 (at least at the bottom end opening 313) being slightly less (e.g., 0.1 to 5% less) than the diameter of the artificial lyo-cake 315, and with the artificial lyo-cake being made of compressible material. The artificial lyo-cake 315 can then exert an outward/expansive pressure to stay secure within the modified vial 306.

While not shown in FIG. 3C or FIG. 3D, the artificial lyo-cake 315 may include alternative means for securing the artificial lyo-cake 315 within the modified vial 306. For example, the means for securing the artificial lyo-cake 315 within the modified vial 306 may be a separate cap that fits over both the artificial lyo-cake 315 and the bottom rim 314. Alternatively, the means for securing may include flexible ribs, as discussed in further detail below with reference to FIG. 5.

In some embodiments, the artificial lyo-vial is labeled (block 340 of FIG. 3E), e.g., manually. The label may identify a respective artificial lyo-vial as a particular type of defect (e.g., low-fill, high-fill, meltback, collapsed lyo-cake, lyo-cake with foreign particles, etc.) or as a non-defect sample, for example. The labeling may be physical labeling (e.g., for storage, or for packaging if the artificial lyo-vial is shipped), or may be a data label that corresponds to the artificial lyo-vial. For example, the label may be used for supervised training of a neural network (e.g., when a system similar to AVI system 100 captures an image of the artificial lyo-vial, and the image is used along with the label to train the neural network), or for testing/qualification of an already-trained neural network (or computer vision system, etc.).

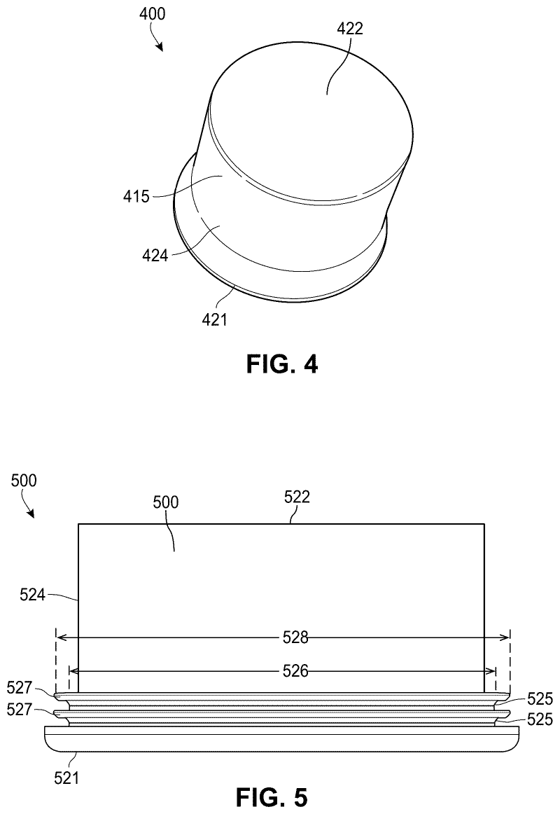

FIG. 4 depicts another artificial lyo-cake 400 having a body portion 415, a base 421, and a top surface 422, with the body portion 415 having an annular surface 424 extending from a perimeter of the base 421 to a perimeter of the top surface 422. The top surface 422 may define a concaved shape, for example. The concave shape defined by the top surface 422 may be similar to, for example, a lyo-cake with a meltback defect. Otherwise, the artificial lyo-cake 400 may be similar to, for example, the artificial lyo-cake 215 of FIG. 2 or the artificial lyo-cake 315 of FIG. 3C.

FIG. 5 depicts an artificial lyo-cake 500 having an annular surface 515, and flexible ribs 527 near a bottom of the artificial lyo-cake 500 but above the base 521. The annular surface 515 has a top surface 522, and extends down to at least the flexible ribs 527. The flexible ribs 527 may be formed of an elastic and/or compressible material (e.g., rubber), have an outside diameter 528 that is larger than the diameter of the annular surface 515, and be generally configured to secure the artificial lyo-cake 500 within an associated modified vial (modified vial 306 of FIG. 3B). The artificial lyo-cake 500 may be similar to, for example, the artificial lyo-cake 215 of FIG. 2, the artificial lyo-cake 315 of FIG. 3C, or the artificial lyo-cake 400 of FIG. 4. Between the ribs 527, the artificial lyo-cake 500 may have a diameter 526 that is less than the rib diameter 528 but slightly larger than the diameter of the annular surface 515 (e.g., if the area 525 and the ribs 527 collectively form a rubber cap that is placed over the remainder of the artificial lyo-cake 500).

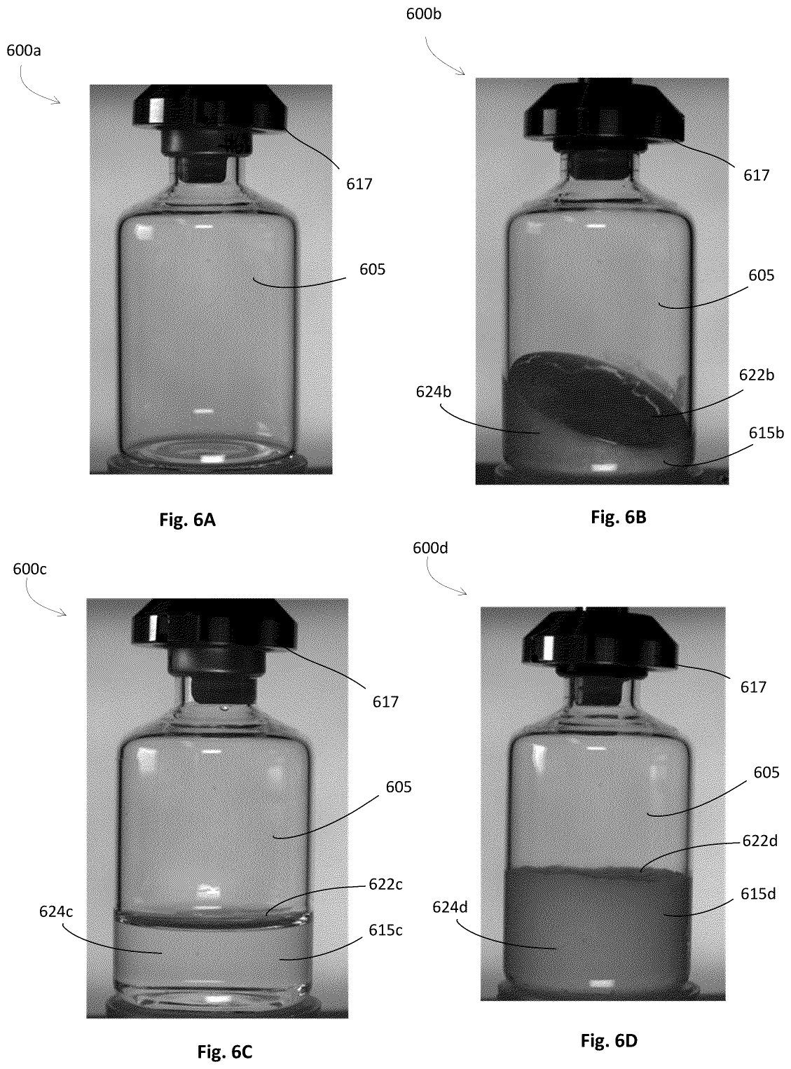

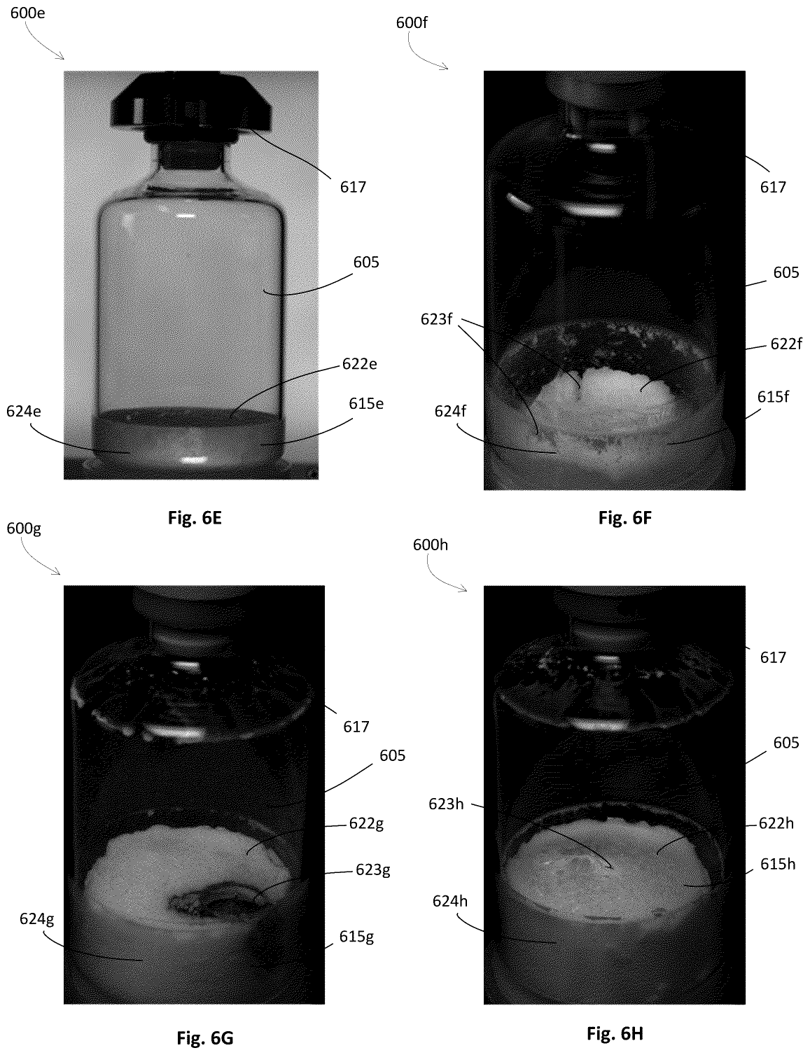

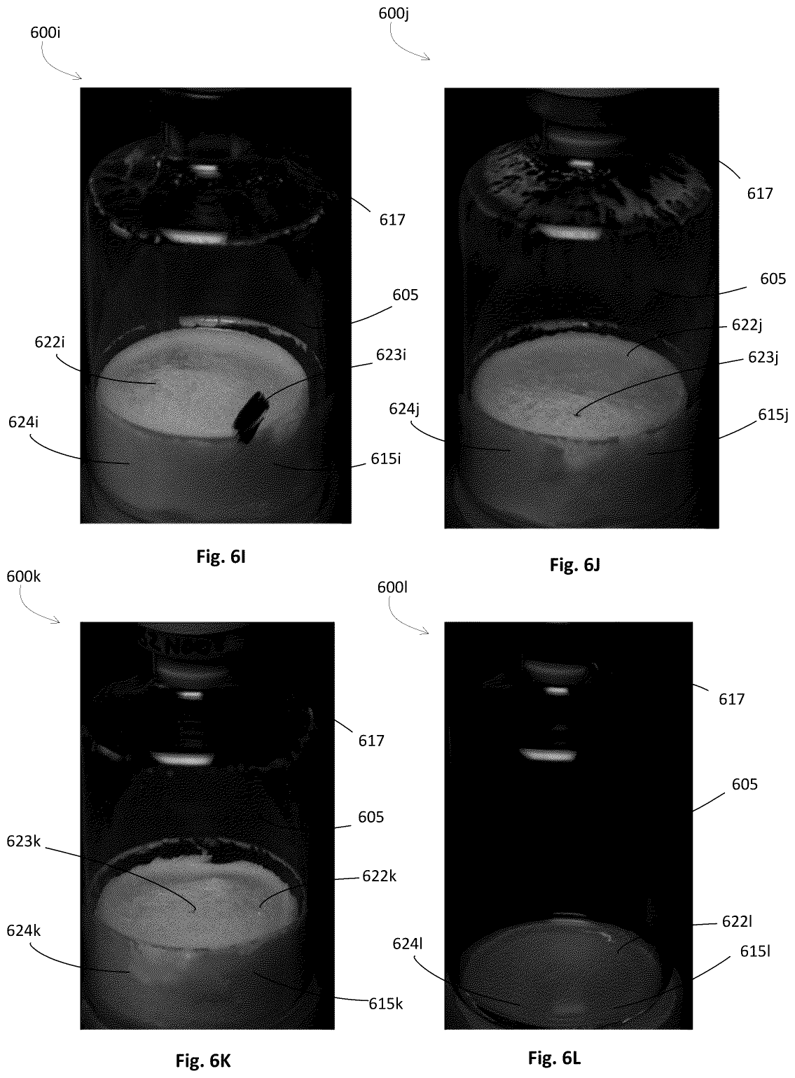

FIGS. 6A through 6L depict greyscale images of example vials with lyophilized product defects 600a-l. The vials illustrated in the images 600a-l are conventional vials that (except for image 600a) contain actual lyophilized product. However, images 600b-l provide examples of what may be artificially recreated using the systems and techniques discussed above.

Referring first to image 600a, a vial 605 (which may be similar to lyo-vial 305, and may be modified to become a vial similar to modified vial 306) includes a seal 617 (e.g., similar to seal 107).

Referring to image 600b, the vial 605 includes a lyophilized product 615b, which has a top surface 622b and an annular surface 624b that reflect a “collapsed cake” defect. An artificial collapsed lyophilized product cake may be generated to replicate the lyophilized product 615b using 3D printing. Optionally, the artificial lyo-cake may be coated with sugar and/or salt diluted with isopropyl alcohol. When creating a replica of a collapsed cake such as lyophilized product 615b, it may be desirable to exceed some threshold slope of the top surface of the cake (e.g., a threshold slope above which the AVI system should reject the sample). Thus, an angle of at least a portion of the top surface of the artificial lyo-cake (e.g., relative to a central axis 106 in FIG. 1) may be set greater than a collapsed cake slope threshold.

Referring to image 600c, the vial 605 includes a lyophilized product 615c, which has a top surface 622c and an annular surface 624c that reflect a “liquefied product” defect. An artificial liquefied lyophilized product cake may be generated using 3D printing and a translucent material. Alternatively, a liquefied lyophilized product cake may be recreated to replicate the lyophilized product 615c using 3D printing. When creating a replica of a liquefied product such as lyophilized product 615c, it may be desirable to coat at least a portion of the 3D printed material with a high-sheen material.

Referring to image 600d, the vial 605 includes a lyophilized product 615d, which has a top surface 622d and an annular surface 624d that reflect a “high fill” defect. An artificial high fill lyophilized product cake may be generated to replicate the lyophilized product 615d using 3D printing. When creating a replica of a high fill defect such as in the lyophilized product 615b, it may be desirable to exceed some threshold cake height (e.g., a threshold height above which the AVI system should reject the sample). Thus, a longitudinal dimension of the artificial lyo-cake may be set greater than a high product fill threshold.

Referring to image 600e, the vial 605 includes a lyophilized product 615e, which has a top surface 622e and an annular surface 624e that reflect a “low fill” defect. An artificial low fill lyophilized product cake may be generated to replicate the lyophilized product 615e using 3D printing. When creating a replica of a low fill defect such as in the lyophilized product 615b, it may be desirable to be below some threshold cake height (e.g., a threshold height below which the AVI system should reject the sample). Thus, a longitudinal dimension of the artificial lyo-cake may be set less than a low product fill threshold.

Referring to image 600f, the vial 605 includes a lyophilized product 615f, which has a top surface 622f and an annular surface 624f, one or both of which may reflect a “collapsed cake” and/or a “meltback” defect 623f. An artificial collapsed lyophilized product may be generated to replicate the lyophilized product 615f using 3D printing. When creating a replica of a collapsed cake or meltback such as in the lyophilized product 615f, it may be desirable to coat at least a portion of a 3D printed artificial lyo-cake with, for example, sugar and/or salt diluted with isopropyl alcohol. It may also be desirable to create one or more discontinuities (e.g., gaps) that are greater than a threshold distance (e.g., a threshold discontinuity length or other distance above which the AVI system should reject the sample). Thus, at discontinuity 623f in at least a portion of the top surface 622f or at least a portion of the annular surface 624f of the artificial lyo-cake may be set greater than a meltback discontinuity threshold distance.

As another alternative, or addition, a discontinuity 623f in the top surface 622f and/or the annular surface 624f color of the artificial lyo-cake 615f may be set greater than a collapsed cake color threshold and/or a meltback color threshold. In context of the grayscale image 600f of FIG. 6F, a collapsed cake color threshold and/or a meltback color threshold may be based on greyscale values of pixels associated with the image 600f of the discontinuity 623f relative to greyscale values of pixels associated with a remaining portion of the top surface 622f or the annular surface 624f in the image 600f, for example.

Referring to image 600g, the vial 605 includes a lyophilized product 615g, which has a top surface 622g and an annular surface 624g, one or both of which may reflect a “colored cake” defect 623g (the non-uniform color not being apparent in the grayscale image of FIG. 6G). An artificial colored lyophilized product cake may be generated to replicate the lyophilized product 615g using 3D printing, and by applying color (e.g., dye) to an area of the artificial lyo-cake after printing.

Referring to image 600h, the vial 605 includes a lyophilized product 615h, which has a top surface 622h and an annular surface 624h that reflect a “fiber particle” (e.g., 100 um, 200 um, 300 um, 400 um, 500 um, 750 um, 1000 um and/or 2000 um fiber particles) defect 623h. An artificial lyophilized product cake with fiber particles visually similar to fiber particles 623h may be generated to replicate the lyophilized product 615h using 3D printing, and by applying fiber particles visually similar to fiber particles 623h to the 3D printed artificial lyo-cake before inserting the artificial lyo-cake into a bottom opening of a modified vial.

Referring to image 600i, the vial 605 includes a lyophilized product 615i, which has a top surface 622i and an annular surface 624i that reflect a “foreign matter” (e.g., unknown foreign matter) defect 623i. An artificial lyophilized product cake with matter visually similar to foreign matter 623i may be generated to replicate the lyophilized product 615i using 3D printing, and by applying matter visually similar to foreign matter 623i before inserting the artificial lyo-cake into a bottom opening of a modified vial. To reproduce more precisely the defect in FIG. 6i (where the defect is partially above the level of the cake), the matter may be applied to the inside surface of the modified vial rather than the artificial lyo-cake.

Referring to image 600j, the vial 605 includes a lyophilized product 615j, which has a top surface 622j and an annular surface 624j, one or both of which reflect a “glass particle” (e.g., 100 um, 200 um, 300 um, 400 um, 500 um, 750 um and/or 1000 um fiber particles) defect 623j. An artificial lyophilized product cake with glass particles visually similar to glass particles 623j may be generated to replicate the lyophilized product 615j using 3D printing, and by applying the glass particles to the artificial lyo-cake before inserting the artificial lyo-cake into a bottom opening of a modified vial.

Referring to image 600k, the vial 605 includes a lyophilized product 615k, which has a top surface 622k and an annular surface 624k that reflect a “metal particles” (e.g., 100 um, 200 um, 300 um, 400 um, 500 um, 750 um and/or 1000 um metal particles) defect 623k. An artificial lyophilized product cake with metal particles visually similar to metal particles 623k may be generated to replicate the lyophilized product 615k using 3D printing, and by applying the metal particles to the 3D printed artificial lyo-cake before inserting the artificial lyo-cake into a bottom opening of a modified vial.

Referring to image 600l, the vial 605 includes a lyophilized product 615l, which has a top surface 622l and an annular surface 624l, one or both of which may reflect an “unusual appearance” defect. An artificial lyophilized product cake with an unusual appearance may be generated to replicate the lyophilized product 615l using 3D printing, and by coating at least a portion of the 3D printed artificial lyo-cake with sugar and/or salt diluted with isopropyl alcohol, or with a high-gloss coating, for example.

Although the systems, methods, devices, and components thereof, have been described in terms of exemplary embodiments, they are not limited thereto. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent that would still fall within the scope of the claims defining the invention.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. An artificial lyo-vial representative of at least a portion of a lyophilized product sample, the artificial lyo-vial comprising:
a portion of a vial, the portion of the vial having a vial wall with a top end opening and a bottom end opening, the bottom end opening having an inside diameter that is greater than an inside diameter of the top end opening, and at least a portion of the vial wall being translucent; and
an artificial lyo-cake secured within the portion of the vial, the artificial lyo-cake having a base, an annular surface, a top surface, a longitudinal dimension extending from the base to at least a portion of the top surface, and an outside diameter that is greater than the inside diameter of the top end opening.

2. The artificial lyo-vial of claim 1, comprising:
a means for securing the artificial lyo-cake within the portion of the vial.

3. The artificial lyo-vial of claim 2, wherein the means for securing the artificial lyo-cake within the portion of the vial includes a separate cap that fits over the artificial lyo-cake and a bottom rim of the portion of the vial.

4. The artificial lyo-vial of claim 2, wherein the means for securing the artificial lyo-cake within the portion of the vial includes a friction fit between the artificial lyo-cake and the portion of the vial.

5. The artificial lyo-vial of claim 4, wherein the friction fit is created by the outside diameter of the artificial lyo-cake being larger than the inside diameter of the bottom end opening of the portion of the vial.

6. The artificial lyo-vial of claim 4, wherein the artificial lyo-cake is formed from a compressible material.

7. The artificial lyo-vial of claim 2, wherein the means for securing the artificial lyo-cake within the portion of the vial includes at least one circumferentially extending flexible rib above the base.

8. A method of manufacturing an artificial lyo-vial, the method comprising:
providing a vial, the vial including a top end opening having a top inside diameter;
creating a bottom end opening by removing at least a portion of a bottom end of the vial, the bottom end opening having a bottom inside diameter that is greater than the top inside diameter;
providing an artificial lyo-cake having a base, a top surface, an annular surface extending between the base and the top surface, and an outside diameter that is greater than the top inside diameter; and
inserting at least a portion of the artificial lyo-cake through the bottom end opening.

9. The method of claim 8, comprising:
providing a means for securing the artificial lyo-cake within the vial.

10. The method of claim 9, wherein providing the means for securing the artificial lyo-cake within the modified vial includes fitting a separate cap over the artificial lyo-cake and a bottom rim of the vial.

11. The method of claim 9, wherein the outside diameter of the artificial lyo-cake is larger than the bottom inside diameter.

12. The method of claim 11, wherein providing the artificial lyo-cake includes forming the artificial lyo-cake from a compressible material.

13. The method of claim 9, wherein providing the means for securing the artificial lyo-cake within the portion of the vial includes at least one circumferentially extending flexible rib above the base.

14. The method of claim 8, wherein the providing the artificial lyo-cake includes printing the artificial lyo-cake using a three dimensional printer.

15. The method of claim 8, further comprising:
coating at least a portion of the artificial lyo-cake prior to inserting at least the portion of the artificial lyo-cake through the bottom end opening.

16. The method of claim 15, wherein coating at least the portion of the artificial lyo-cake includes coating at least the portion of the artificial lyo-cake with sugar diluted with isopropyl alcohol.

17. The method of claim 15, wherein coating at least the portion of the artificial lyo-cake includes coating at least the portion of the artificial lyo-cake with salt diluted with isopropyl alcohol.

18. The method of claim 8, further comprising:
applying a foreign particle to the artificial lyo-cake prior to inserting at least the portion of the artificial lyo-cake through the bottom end opening.

19. The method of claim 18, wherein applying the foreign particle to at least the portion of the artificial lyo-cake includes applying at least one fiber particle.

20. The method of claim 18, wherein applying the foreign particle to at least the portion of the artificial lyo-cake includes applying at least one metal particle.

21. The method of claim 18, wherein applying the foreign particle to at least the portion of the artificial lyo-cake includes applying at least one glass particle.

22. A method of manufacturing an artificial lyo-cake, the method comprising:
generating three-dimensional data that defines a base of a lyo-cake, an annular surface of the lyo-cake, and a top surface of the lyo-cake;
receiving the three-dimensional data at a controller of a three-dimensional printer;
controlling, using the controller, material discharge from a material discharge device of the three-dimension printer to dispense a material based on at least z-values of the three-dimensional data; and
controlling, using the controller, a position of the material discharge device relative to a platform based on at least x-values and y-values of the three dimensional data.

23. The method of claim 22, further comprising:
adding label data to the three-dimensional data, wherein the label data is based on a known type of artificial lyo-cake defect represented by the three-dimensional data.

24. The method of claim 22, wherein the generating the three-dimensional data includes setting the longitudinal dimension to be less than a low product fill threshold.

25. The method of claim 22, wherein the generating the three-dimensional data includes setting the longitudinal dimension to be greater than a high product fill threshold.

26. The method of claim 22, wherein an angle of at least a portion of the top surface, relative to a horizontal plane that is orthogonal to a central axis of the portion of the vial, is greater than at least one of: a meltback angle threshold or a collapsed cake angle threshold.

27. The method of claim 22, wherein a discontinuity in at least a portion of the top surface or at least a portion of the annular surface is greater than at least one of: a meltback discontinuity threshold distance or a collapsed cake discontinuity threshold distance.

28. The method of claim 22, wherein a discontinuity in a surface texture is greater than a meltback color threshold.

29. The artificial lyo-vial of claim 22, wherein a discontinuity in a color of the top surface or the annular surface is greater than a collapsed cake color threshold.

30.-42. (canceled)
